SEQUENCE LISTING (16 sequences)

SEQ ID NO: 1 - DNA, 326 bp, Artificial Sequence
  HCV 40..372: corresponds to IRES sequence of HCV
ctcccctgtgaagaactactgtcttcacgcagaaagcgtctagccatggcgttagtatgagtgtcgtgcagcctccaggaccccccctcccgggagagccatagtggtctgcggaaccggtgagtacaccggaattgccaggatgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaagaaaaa

SEQ ID NO: 2 - DNA, 80 bp, Artificial Sequence
  HCV 40..119: corresponds to a portion (region II) of HCV IRES sequence
ctcccctgtgaggaactactgtcttcacgcagaaagcgtctagccatggcgttagtatgagtgttgtgcagcctccagga

SEQ ID NO: 3 - DNA, 37 bp, Artificial Sequence
  HCV 56..92: corresponds to a portion (consensus sequence) of HCV IRES sequence
tactgtcttcacgcagaaagcgtctagccatggcgtt

SEQ ID NO: 4 - protein, 814 aa, Artificial Sequence
  p116 1..814: corresponds to p116 subunit of eIF3
MQDAENVAVPEAAEERAEPGQQQPAAEPPPAEGLLRPAGPGAPEAAGTEASSEEVGIAEAGPEPEVRTEPAAEAEAASGPSESPSPPAAEELPGSHAEPPVPAQGEAPGEQARDERSDSRAQAVSEDAGGNEGRAAEAEPRALENGDADEPSFSDPEDFVDDVSEEELLGDVLKDRPQEADGIDSVIVVDNVPQVGPDRLEKLKNVIHKIFSKFGKITNDFYPEEDGKTKGYIFLEYASPAHAVDAVKNADGYKLDKQHTFRVNLFTDFDKYMTISDEWDIPEKQPFKDLGNLRYWLEEAECRDQYSVIFESGDRTSIFWNDVKDPVSIEERARWTETYVRWSPKGTYLATFHQRGIALWGGEKFKQIQRFSHQGVQLIDFSPCERYLVTFSPLMDTQDDPQAIIIWDILTGHKKRGFHCESSAHWPIFKWSHDGKFFARMTLDTLSIYETPSMGLLDKKSLKISGIKDFSWSPGGNIIAFWVPEDKDIPARVTLMQLPTRQEIRVRNLFNVVDCKLHWQKNGDYLCVKVDRTPKGTQGVVTNFEIFRMREKQVPVDVVEMKETIIAFAWEPNGSKFAVLHGEAPRISVSFYHVKNNGKIELIKMFDKQQANTIFWSPQGQFVVLAGLRSMNGALAFVDTSDCTVMNIAEHYMASDVEWDPTGRYVVTSVSWWSHKVDNAYWLWTFQGRLLQKNNKDRFCQLLWRPRPPTLLSQEQIKQIKKDLKKYSKIFEQKDRLSQSKASKELVERRRTMMEDFRKYRKMAQELYMEQKNERLELRGGVDTDELDSNVDDWEEETIEFFVTEEIIPLGNQE

SEQ ID NO: 5 - protein, 106 aa, Artificial Sequence
  p116 175..279: corresponds to a portion (RRM) of eIF3 p116 subunit
MDRPQEADGIDSVIVVDNVPQVGPDRLEKLKNVIHKIFSKFGKITNDFYPEEDGKTKGYIFLEYASPAHAVDAVKNADGYKLDKQHTFRVNLFTDFDKYMTISDEW

SEQ ID NO: 6 - DNA, 33 bp, Artificial Sequence
  primer_bind 1..33: HCV RRM 5′ primer (RRMfwd)
catatggatcggccccaggaagcagatggaatc

SEQ ID NO: 7 - DNA, 33 bp, Artificial Sequence
  primer_bind 1..33: HCV RRM 3′ primer (RRMrev)
gtgctcgagccactcgtcactgatcgtcatata

SEQ ID NO: 8 - DNA, 29 bp, Artificial Sequence
  primer_bind 1..29: HCV IRES 5′ primer (IRESfwd)
accgctagcctcccctgtgaggaactact

SEQ ID NO: 9 - DNA, 46 bp, Artificial Sequence
  primer_bind 1..46: HCV IRES 3′ primer (IRESrev)
gaaagcttttttctttgaggtttaggatttgtgctcatgatgcacg

SEQ ID NO: 10 - DNA, 95 bp, Artificial Sequence
  primer_bind 1..95: primer IIIabcfwd which corresponds to T7 polymerase promoter + 139-215 of HCV (regions IIIa-IIIb)
taatacgactcactatagggtagtggtctgcggaaccggtgagtacaccggaattgccaggacgaccgggtcctttcttggataaacccgctcaa

SEQ ID NO: 11 - DNA, 60 bp, Artificial Sequence
  primer_bind 1..60: primer IIIabcrev which corresponds to 193-252 of HCV (regions IIIb-IIIc)
tagcagtctcgcgggggcacgcccaaatctccaggcattgagcgggttgatccaagaaag

SEQ ID NO: 12 - DNA, 20 bp, Artificial Sequence
  primer_bind 1..20: primer T7 which corresponds to a portion of primer IIIabcfwd
taatacgactcactataggg

SEQ ID NO: 13 - DNA, 21 bp, Artificial Sequence
  primer_bind 1..21: primer which corresponds to a portion of primer IIIabcrev
tagcagtctcgcgggggcacg

SEQ ID NO: 14 - DNA, 22 bp, Artificial Sequence
  primer_bind 1..22: primer SP6
tatttaggtgacactatagaat

SEQ ID NO: 15 - DNA, 27 bp, Artificial Sequence
  primer_bind 1..27: primer Linkerrev
gtcctggtggctgcaggacactcatac

SEQ ID NO: 16 - DNA, 48 bp, Artificial Sequence
  primer_bind 1..48: primer LinkerSP6
tatttaggtgacactatagaatactcaagctatgcatccaacgcgttg

(19) United States
(12) Patent Application Publication
(10) Pub. No.: US 2006/0035212 A1
(43) Pub. Date: Feb. 16, 2006

(54) Molecules inhibiting hepatitis c virus protein synthesis and method for screening same

(75) Inventor: Larissa Balakireva, Grenoble (FR)

Correspondence Address: HESLIN ROTHENBERG FARLEY & MESITI PC, 5 COLUMBIA CIRCLE, ALBANY, NY, 12203, US

(73) Assignee: Universte Joseph Fourier, Grenoble Cedex 9 (FR)

(21) Appl. No.: 10/538,471

(22) PCT Filed: Dec. 11, 2003
(86) PCT No.: PCT/FR03/03675

§ 371 Date: Jun. 3, 2005

(30) Foreign Application Priority Data
Dec. 12, 2002 (FR) 02/15718

(51) Int. Cl. C12Q 1/70

(52) U.S. Cl. 435/5

(57) ABSTRACT

The invention concerns a method for screening molecules which consists, in vitro: a) in incubating together the subunit p116 (SEQ ID4) of the elF3 protein, the nucleotide sequence of region II (SEQ ID2) of the HCV IRES or any sequence containing at least 10 successive nucleotides of the region II (SEQ ID2) of the HCV IRES and the molecule to be tested; b) in detecting the possible formation of p116/IRES region II complex, the absence of complex indicating the inhibiting capacity of the tested molecule, to inhibit the formation of said complexes; c) in selecting the molecules inhibiting the formation of complexes.

The invention relates to the treatment of viral or nonviral pathologies which involve proteins whose synthesis is initiated by means of an internal ribosome entry site (IRES), at least part of the sequence of which is similar from one IRES to another. Among these pathologies are in particular, but without implying limitation, among viral pathologies, viruses belonging to the family Flaviridae such as the hepatitis C virus (HCV), the classical swine fever virus (CSFV) or the bovine viral diarrhea virus (BVDV), and among nonviral pathologies, cancers in which certain proteins are involved, for instance fibroblast growth factors responsible for the neovascularization of developing tumors, the c-myc proto-oncogene, etc. More precisely, the treatment proposed in the invention consists in preventing the binding of the translation initiation factor, eIF3, to the RNA constituting the 5′ noncoding portion of the IRES (Internal Ribosome Entry Site) sequence of viral genomes or of certain genes involved in the abovementioned pathologies, so as to inhibit protein synthesis. Consequently, a subject of the invention is also a method for screening molecules capable of inhibiting the formation of the complex: IRES sequence/eIF3, in particular nucleotides 56 to 92 of domain II of the IRES and the recombinant polypeptide corresponding to the central portion (amino acids 185 to 279) of the p116 protein subunit (also called p110, eIF3b, eIF3eta (BLAST P55884)) of eIF3.

The process of research and development of novel therapeutic molecules, drug discovery, requires, first and foremost, the identification of novel targets associated with diseases (protein, RNA or DNA, or complexes thereof) and validation thereof. The target identified and validated is then used in tests for screening molecules, which make it possible to select active molecules. This is the approach that is proposed by the applicant, the target consisting of a sequence specific for the IRES of HCV (domain II) and a recombinant polypeptide derived from the sequence of the p116 subunit of eIF3.

In the remainder of the description, the invention is more particularly described in relation to the treatment of the HCV virus, although it also applies to the classical swine fever virus (CSFV) or the bovine viral diarrhea virus (BVDV), given the strong homology that exists between these viruses belonging to the same family.

The hepatitis C virus has been identified as being responsible for the non-A, non-B hepatitis commonly developed during chronic malignant pathologies, such as for example liver cirrhosis or else hepatocellular carcinoma. HCV is transmitted by blood transfusion or transfusion of blood derivatives. The HCV genome is in the form of a single-stranded RNA in the region of 9.4 kb in size and encoding a single polyprotein consisting of 3010 amino acids (Choo et al., 1989).

Unlike the conventional scheme, the initiation of translation of the HCV messenger RNA does not take place by CAP recognition (“cap-dependent” translation), since said cap is absent, but by means of an internal ribosome entry site (IRES) positioned in the 5′ untranslated region (5′-UTR) of HCV, between nucleotides 40 and 372 of the HCV sequence (cap-independent translation) (equivalent to SEQ ID 1 according to the invention). Since the mechanism of synthesis of viral proteins is very different from that of the host cell, a possible strategy for developing novel therapeutic molecules consists in inhibiting viral protein synthesis without having any influence on the host cell's protein synthesis. In addition, since the sequence of the IRES is a very conserved region in this virus that is reputedly very variable (92% homology), it may be expected that the use of this sequence as a target would be particularly advantageous.

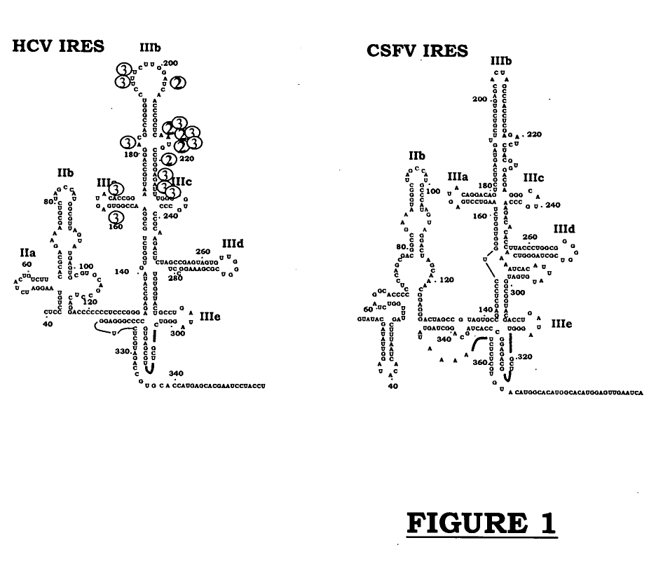

Various structural studies have shown that the HCV IRES is folded on itself so as to form three looped domains or regions, respectively regions II (IIa, IIb), III (IIIa, IIIb, IIIc, IIId, IIIe, IIIf) and IV as represented in FIG. 1 (Zhao et al, 2001), the IRES also comprising an AUG start codon. The single-stranded RNA of CSFV and of BVDV also contains an IRES sequence containing an AUG start codon, the structure of the IRES being similar to that of HCV (FIG. 1). In addition, alignment of the sequences consisting of the genome of these three viruses shows strong homology of region II of the IRES, in particular of the RRM (RNA recognition motif) site, which tends to imply that molecules which act on the IRES of HCV could also act on that of CSFV or of BVDV.

In practice, the initiation of translation of the mRNA begins with the recognition and binding, by the IRES, of the 40S ribosomal subunit and of initiation factors, in particular the initiation factor called “eIF3”.

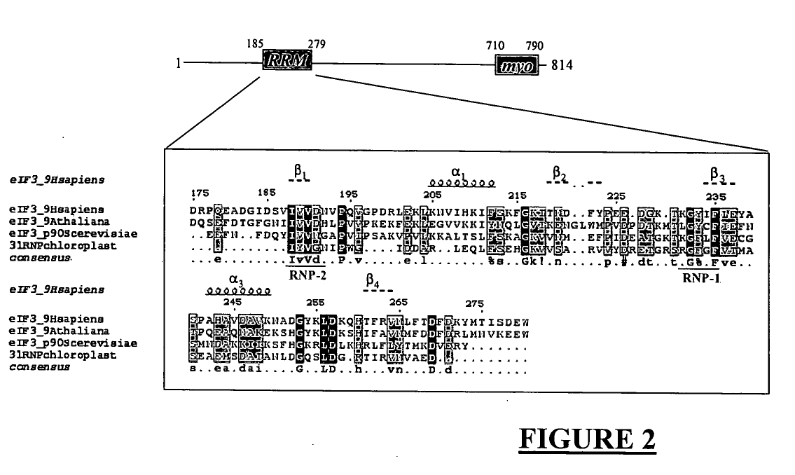

The initiation factor eIF3 is a multiprotein complex consisting of 10 different subunits such as, for example, p36, p44, p47, p66, p110, p116 and p170. Studies to predict secondary structure have shown that the p116 subunit has, in its central portion, located between amino acids 185 and 279, an RNA recognition motif (RRM) β-α-β-β-α-β. The location of the eIF3 p116 subunit recognition motif is represented in FIG. 2.

Similarly, the C-terminal portion of the p44 subunit has, itself also, a similar structure β-α-β-β-α-β, corresponding to a hypothetical RRM. This type of motif is found in a large number of RNA-binding proteins, for instance the hnRNP or snRNP proteins, but also in some single-stranded DNA-binding proteins. According to the algorithms for predicting secondary structure, the central portion of the p116 subunit is folded according to a conformation similar to that of the RRMs that are known for the conserved amino acids IVVD and TK/RGF/YVE located in sheets 1 and 3 corresponding to the recognition motifs RNP-2 and RNP-1 (see FIG. 2). Although, by virtue of its secondary structure and its homology, the RRM of eIF3 p116 satisfies the criteria for putative RNA-binding proteins, its real ability to bind RNA has never been previously demonstrated.

In fact, document FR-A-2 815 358 describes a method for treating hepatitis C consisting in preventing HCV protein synthesis by supposed inhibition of the binding of the eIF3 p116 subunit to region III of the IRES. The candidate molecules for this inhibition correspond to polypeptides exhibiting an affinity with region III of the IRES that is greater than that of the p116 subunit of eIF3. In practice, the polypeptide inhibitors are obtained by screening mutated p116 proteins with the HCV IRES sequence. More precisely, only the central portion corresponding to the recognition motif (RRM) is mutated, the polypeptide being capable of binding to loop IIIb of the HCV IRES with an affinity that is greater than or equal to that of the nonmutated RRM of p116. In practice, the mutations are introduced into the RRM by random mutagenesis or by targeted mutagenesis according to the phase display technique. Here again, no indication is given concerning the nucleotide sequence of region III of the IRES that is capable of interacting with the mutated RRM. In addition, no result of any possible inhibition is given in the examples.

Sizova et al., 1998, have shown that eIF3 protects the apical region IIIb of the IRES of HCV and of CSFV, in particular nucleotides 204, 212, 214, 215 and 220 (see FIG. 1, nucleotides marked \textcircled{2}), against enzymatic cleavage or against chemical modifications. More recently, Kieft et al., 2001, using the same methods as those used by the abovementioned Sizova, have identified the nucleotides of stem IIIa, loop IIIb and stem IIIb as being the main elements of the interaction (see FIG. 1, nucleotides marked \textcircled{3}). In addition, using the “filter-binding assay” technique, these various authors have shown that deletion of the apical region IIIb results in an at least 10-fold decrease in the eIF3-IRES interaction. Thus, the apical loop IIIb is currently considered to be the most probable site for binding of eIF3. However, none of these documents shows precisely the existence of an interaction between the isolated domain IIIb and eIF3. Similarly, none of them identifies a specific eIF3-binding RNA sequence.

Buratti et al., 1998, have shown that the p170 and 116/p110 proteins of eIF3 bind to region III of the HCV IRES, without however, here again, identifying the RNA sequence of the IRES envisioned.

Document Wo 01/44266 also reports the interaction between the p116 subunit of the initiation factor eIF3 and region III of the HCV IRES, more precisely in a domain capable of pairing and defining two nucleotide sequences of 7 bases and 9 bases, respectively. The definition of this minimum motif makes it possible to carry out an assay for identifying compounds capable of competing in the formation of the eIF3-HCV complex.

Moreover, document U.S. Pat. No. 6,001,990 describes a series of oligonucleotides, selected for their ability to inhibit HCV RNA translation. Among these, the 28-nucleotide oligonucleotide of sequence TAGACGCTTTCTGCGTGAAGACAGTAGT, corresponding to the sequence SEQ ID 3 of this document, hybridizes effectively with region II of the HCV IRES.

It is known that the RNA-binding proteins of the RRM family recognize short (<10 nt) single-stranded sequences belonging to a loop in RNA structures of stem-loop type or to a stem extension. These short fragments integrated into an appropriate structural context are essential to the specific binding of RRMs to messenger or premessenger RNAs comprising 1000 nucleotides or more. Consequently, it is important to identify the minimum sequence of the RNA of the IRES that interacts with the RRM. Specifically, the identification of this minimum sequence makes it possible, first of all, to understand the mechanism of the interaction, but also to design complementary antisense oligonucleotides (generally between 30-35 nt in length) capable of inhibiting the formation of the RNA/protein complex or, in the case of iRNA (interfering or silencing RNA between 21-23 nt in length), of targeting the region of interaction. The identification of the minimum sequence is also essential for carrying out the structural studies necessary for the screening in silico, and also for optimizing active molecules. According to a technique known to those skilled in the art, the atomic structure of the RNA/protein complex or RNA alone is sought in 3 dimensions by NMR. It is known that this technique can only be used for short (less than 25 nt) fragments of RNA alone or complexed RNA. A second technique corresponds to X-ray crystallography, which technique can be applied to RNA fragments that are longer, the length nevertheless being limited to 70 nt. Conversely, protein crystallography is not limited by size, but can however only be applied to isolated proteins and not to multiprotein complexes such a eIF3.

In other words, one of the problems that the invention proposes to solve is that of precisely identifying the shortest RNA sequence of the IRES that binds to the p116 RRM, so as to be able to use this sequence in methods for screening molecules of interest.

In the course of its research, the applicant has not only discovered that the p116 subunit of eIF3 does not bind to region III but to region II of the HCV IRES (SEQ ID 2 according to the invention), but also succeeded in precisely identifying the nucleotide sequence of the IRES, subsequently referred to as consensus sequence (SEQ ID 3 according to the invention), that interacts with the p116 RRM.

Moreover, the functionality of the RRM (SEQ ID 5 according to the invention) predicted in the p116 subunit of eIF3 (SEQ ID 4 according to the invention) has been demonstrated by the applicant. Thus, the expression of this central portion of the p116 subunit of eIF3 in recombinant form and the demonstration of its specific binding to domain II of the HCV IRES makes it possible to use this polypeptide instead of the eIF3 multiprotein complex, that requires purification from a lysate of cells in culture or of reticulocytes. This makes it possible, firstly, to make the screening much less expensive, but especially to apply structural biology methods, such as NMR or crystallography, in order to solve the atomic structure of this RNA-protein complex and to design inhibitors.

Given the homology that exists between the IRES sequence of HCV and those of CSFV and BVDV, the discovery of the consensus sequence makes it possible to envision treating the various pathologies in which these viruses are involved by virtue of molecules capable of blocking protein synthesis by inhibition of the binding of the p116 protein subunit of eIF3, in particular of its RRM, to region II of these viruses.

The candidate molecules may be existing or future molecules whose inhibitory properties are tested by screening.

Consequently, the invention relates first of all to a method for screening molecules according to which, in vitro:
a/ the p116 subunit (SEQ ID 4) of the eIF3 protein, the nucleotide sequence of region II (SEQ ID 2) of the HCV IRES or any sequence containing at least 10 successive nucleotides of region II (SEQ ID 2) of the HCV IRES, and the molecule to be tested are incubated together,
b/ the possible formation of p116/IRES region II complexes is then detected, an absence of complex reflecting the inhibitory capacity of the molecule tested, to inhibit the formation of said complexes,
c/ the molecules that inhibit the formation of the complexes are selected.

The term “molecule” denotes any known or future chemical molecule of synthetic or natural origin. This term also denotes multiprotein complexes such as antibodies, proteins, peptides, ribonucleotides or deoxyribonucleotides, that may be natural or modified, and PNA (peptide nucleic acid) molecules.

As already mentioned, the p116 subunit of eIF3 contains an RNA recognition motif (RRM) located in the central portion, more specifically between amino acids 175 and 279 of the sequence SEQ ID 4. The amino acid sequence of the RRM of p116 corresponds to the sequence SEQ ID 5.

In other words, and in an advantageous embodiment of the screening method of the invention, only the sequence of the recognition motif of the p116 protein (SEQ ID 5) is incubated. The p116 RRM polypeptide (SEQ ID 5) is preferably produced in recombinant form, in combination with a tag that facilitates its purification. It may be labeled, during preparation, with radioactive, biotinylated or fluorescent amino acids, for detecting the formation of the protein/RNA complex.

Moreover, and as will be demonstrated in the examples, the applicant has precisely identified the sequence of region II of the HCV IRES that binds to the recognition motif of the p116 protein. This sequence, referred to in the remainder of the description as “consensus sequence” (SEQ ID 3), contains 37 nucleotides located between nucleotides 56 and 92 of the HCV IRES sequence. This sequence can be introduced by chemical synthesis or by in vitro transcription, and labeled by radioactivity, biotinylation or fluorescence.

Consequently and in a preferred embodiment, only part of region II is incubated, and corresponds to the consensus nucleotide sequence SEQ ID 3 or a sequence comprising at least 8 successive nt of the sequence SEQ ID 3.

Since RNA is an unstable molecule, the RNA molecule to be incubated according to the invention can be modified for the purpose of increasing its stability. With this aim, it can contain phosphorothioate, methyl phosphonate, phosphoramidate, acetamidate, carbamate, etc., backbones. It can also contain modified bases, such as 2′-deoxynucleosides, 2′-O-alkylnucleosides or 2′-fluoro-2′-deoxynucleosides.

In practice, the incubation is carried out in a buffer solution at ambient temperature. Advantageously, increasing concentrations of molecules to be tested are incubated in order to detect a possibly dose-dependent effectiveness.

The second step of the method consists in detecting the formation of protein/RNA complexes. Any detection method known to those skilled in the art can be used. If radiolabeled RNA is used, the RNA/protein/molecule mixture is advantageously filtered through a nitrocellulose membrane, and the detection is then carried out by measuring the radioactivity attached to the membrane, corresponding to the amount of RNA bound to the protein. Alternatively, the RNA can be non-radioactively labeled (for example with biotin), incubated with the protein, filtered through a nitrocellulose membrane and visualized using streptavidin or specific antibodies.

Other techniques can be used to detect the RNA/protein interaction, such as SPA (Scintillation Proximity Assay), FRET (Fluorescence. Resonance Energy Transfer), HTRF (Homogeneous Time-Resolved Fluorescence), LANCE (Lanthanide Chelation Excitation), FP (Fluorescence Polarization), FCS (Fluorescence Correlation Spectroscopy) or FL (Fluorescence Lifetime Measurements).

In the SPA approach, the purified p116 RRM polypeptide is immobilized using antibodies specific for the myc epitope, present in the C-terminal portion of the polypeptide, or using Ni²⁺ chelating agents, on a 96-well plate impregnated with scintillant. The radiolabeled consensus RNA is added. A signal is only detected if the RNA is bound to the immobilized polypeptide.

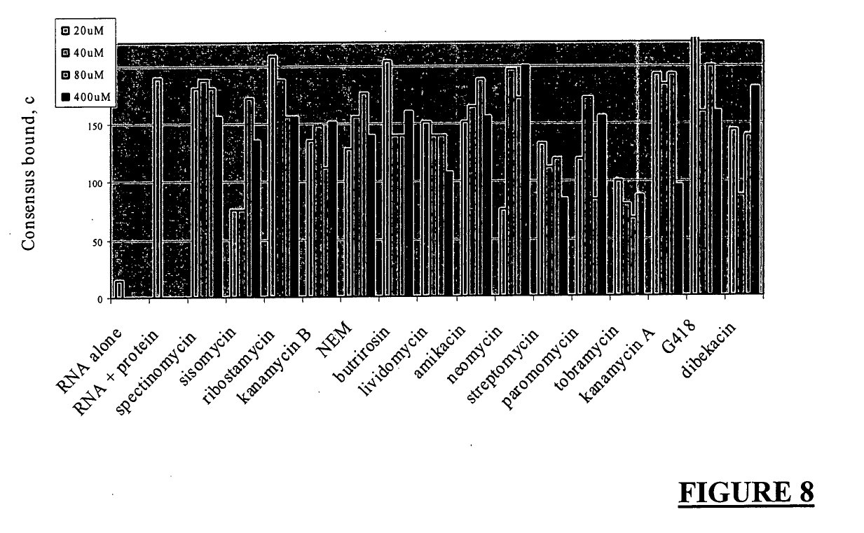

As will be described in the examples (FIG. 8), this screening technique was used by the applicant to study and compare the ability of 15 different aminoglycosides to dissociate the p116 RRM/consensus RNA complex.

In an advantageous embodiment, the results of the screening according to the invention are correlated with those of additional assays consisting in testing, ex vivo, the influence of the selected molecule on cap-independent translation (IRES-dependent) and cap-dependent translation using a bicistronic construct.

This step can be carried out by any method known to those skilled in the art, in particular by constructing bicistronic vectors consisting of two luciferases framing the sequence of region II (SEQ ID 2) or any sequence containing at least 10 successive nucleotides of region II (SEQ ID 2), or the consensus sequence (SEQ ID 3) or a flanking sequence comprising at least 8 successive nucleotides of the sequence SEQ ID 3; the first luciferase being translated in a cap-dependent manner and the second in a cap-independent manner, or vice versa. Cells are then transfected with the bicistronic vectors, and the rate of translation with Dual Luciferase is then measured. The cells capable of being transfected are selected conventionally by those skilled in the art, for instance HeLa cells or else Huh 7 cells.

Comparison of the results obtained with the screening assay proposed by the applicant and those of the bicistronic assays makes it possible to select only the molecules capable both of preventing the binding of p116 to domain II of the IRES in vitro and of specifically inhibiting the IRES-dependant translation in a cell model (ex vivo). In addition, as shown in example 4, the fact that tobramycin is both capable of dissociating the p116/II complex in vitro, at all the concentrations tested, and is the most specific inhibitor of the IRES-controlled translation demonstrates the validity and the relevance of the choice of the p116/IRES complex as a screening target.

Consequently, the invention also relates to the use of the molecules identified at the end of the screening method described above, for preparing a medicinal product intended for the treatment of hepatitis C (HCV), of classical swine fever (CSFV), or of bovine viral diarrhea (BVDV).

More broadly, any molecule capable of inhibiting, in vitro, binding of the p116 protein, in particular its recognition motif (RRM), to region II or a sequence containing at least 10 successive nucleotides of region II (SEQ ID 2), in particular a part of region II corresponding to the sequence SEQ ID 3 or a sequence comprising at least 8 successive nucleotides of the sequence SEQ ID 3, can be used for producing a medicinal product intended for the treatment of hepatitis C(HCV), of classical swine fever (CSFV), or of bovine viral diarrhea (BVDV).

In the context of a first trial using the screening method of the invention, the applicant noted that aminoglycosides, in particular tobramycin, was capable of inhibiting the binding of the RRM of p116 to the consensus sequence of region. II of the IRES and that, in addition, this inhibition did not affect the cap-dependant translation.

Consequently, the invention also relates to the use of aminoglycosides, in particular of tobramycin, for producing a composition intended for the treatment of hepatitis C (HCV), of classical swine fever (CSFV) or of bovine viral diarrhea (BVDV).

This may also involve aminoglycoside derivatives, in particular tobramycin derivatives, having properties that are improved in pharmaceutical terms. These aminoglycosides, preferably tobramycin, or their derivatives, can be administered in combination with liposomes for the purpose of better absorption.

In particular, the amino group in the 6′-position in tobramycin is particularly exposed and can be selectively acetylated and then used to graft other groups. The invention therefore also relates to the use of tobramycin analogues, particularly those modified in the 6′ amino position, for the treatment of hepatitis C.

Moreover, the discovery of the consensus sequence makes it possible to use an antisense oligonucleotide complementary to the sequence SEQ ID 3 or any sequence comprising at least 8 successive nucleotides of the sequence SEQ ID 3, with the exception of the sequence TAGACGCTTTCTGCGTGAAGACAGTAGT, as a medicinal product, in particular for the treatment of hepatitis C(HCV), of classical swine fever (CSFV), or of bovine viral diarrhea (BVDV). Along the same lines, iRNAs containing 19 nucleotides of the sequence SEQ ID 3 (consensus sequence) flanked with UU can be used as a medicinal product for the treatment of the same pathologies as above.

In a first embodiment, a subject of the invention is therefore also a pharmaceutical composition comprising an antisense oligonucleotide complementary to the sequence SEQ ID 3 or any sequence comprising at least 8 successive nucleotides of the sequence SEQ ID 3, with the exception of the sequence TAGACGCTTTCTGCGTGAAGACAGTAGT.

As already mentioned, the molecules tested in the screening method may be known molecules, for instance aminoglycosides, but also molecules that remain to be developed.

In the latter case, it appears to be possible to identify, in silico, using a molecule library, molecules capable of inhibiting the protein synthesis of viruses belonging to the family Flaviridae.

Consequently, the invention also relates to a method for screening a molecule library, in silico, consisting:
in determining the atomic coordinates either of region II of the IRES (SEQ ID 2) of HCV or of any sequence containing at least 10 successive nucleotides of region II (SEQ ID 2) of the IRES of HCV, or of the sequence that binds specifically to the RRM of the p116 protein of eIF3 (SEQ ID 3) or a sequence comprising at least 8 successive nucleotides of the sequence SEQ ID 3, or of the complex of region II (SEQ ID 2) or of the specific sequence (SEQ ID 3) with the recognition motif of the p116 protein of eIF3 (SEQ ID 5),
and then in screening the chemical molecule library with the atomic coordinates thus determined.

Any software known to those skilled in the art can be used for determining the atomic coordinates.

The molecules thus identified may then be tested in the method described above, consisting in detecting, in vitro, RNA/protein complexes.

The invention and the advantages that ensue therefrom will emerge more clearly from the following implementation example in support of the attached figures.

FIG. 1 is a representation of the structure of the HCV IRES. It consists of 3 loop domains, II (IIa, IIb), III (IIIa, IIIb, IIIc, IIId, IIIe, IIIf) and IV. The references \textcircled{2} and \textcircled{3} indicate the nucleotides involved in the binding of eIF3 according to the references Sizova et al. (1998) and Kieft et al. (2001), respectively.

FIG. 2 shows the location of the RNA recognition motif in the p116 subunit of eIF3 and the prediction of its secondary structure.

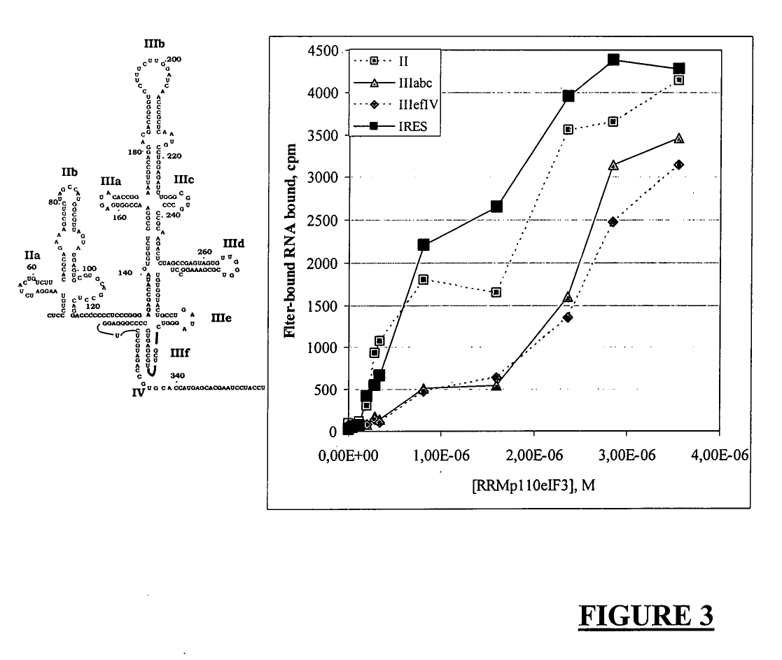

FIG. 3 compares the affinity of the RRM of p116 for regions II, IIIabc, IIIefIV and the entire IRES of HCV, measured after retention on nitrocellulose. To the left, a graphic representation of the HCV IRES makes it possible to locate the various fragments tested.

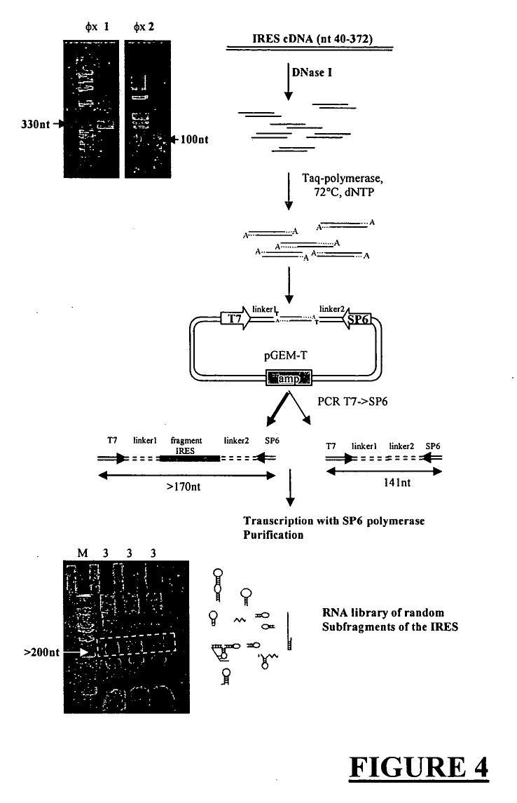

FIG. 4 is a scheme showing the principle of the method for producing random subfragments of the HCV IRES.

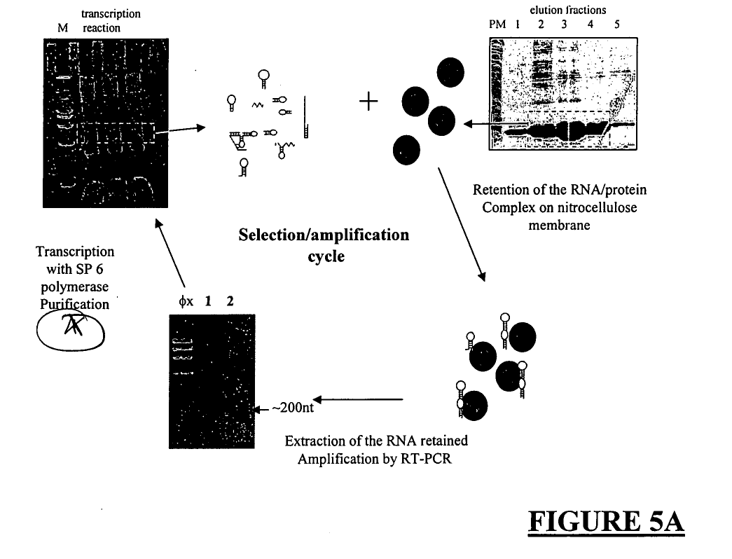

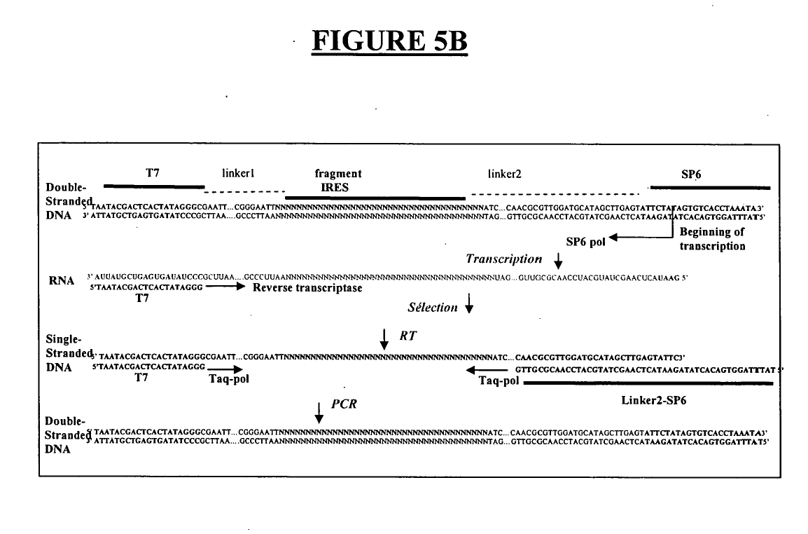

FIG. 5 is a scheme showing the principle of the method for selecting the random subfragments specific for the RRM of eIF3 p116, obtained according to the scheme of FIG. 4 (5A), and the sequences of the transcription templates and of the primers used (FIG. 5B).

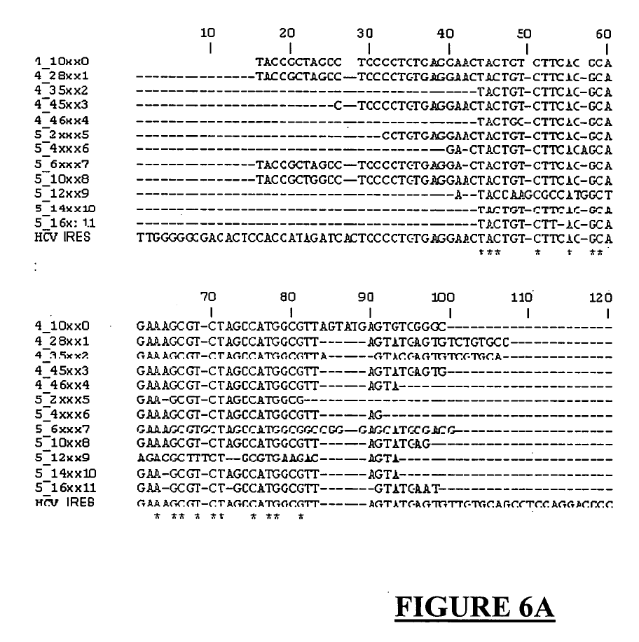

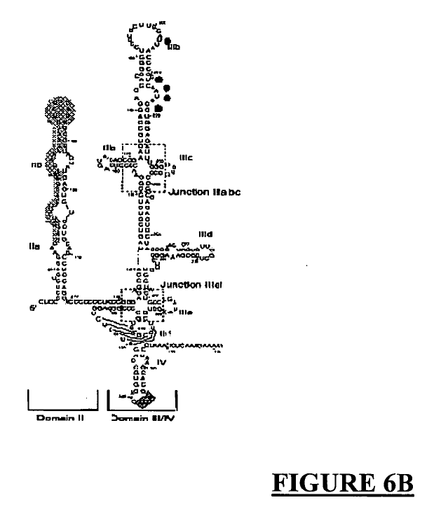

FIG. 6 represents the results of alignment of the RNA sequences selected at the end of the 4^{th} and 5^{th} selection/amplification cycles (6A), and the location of the “consensus” sequence (sense orientation) in the IRES of HCV (6B).

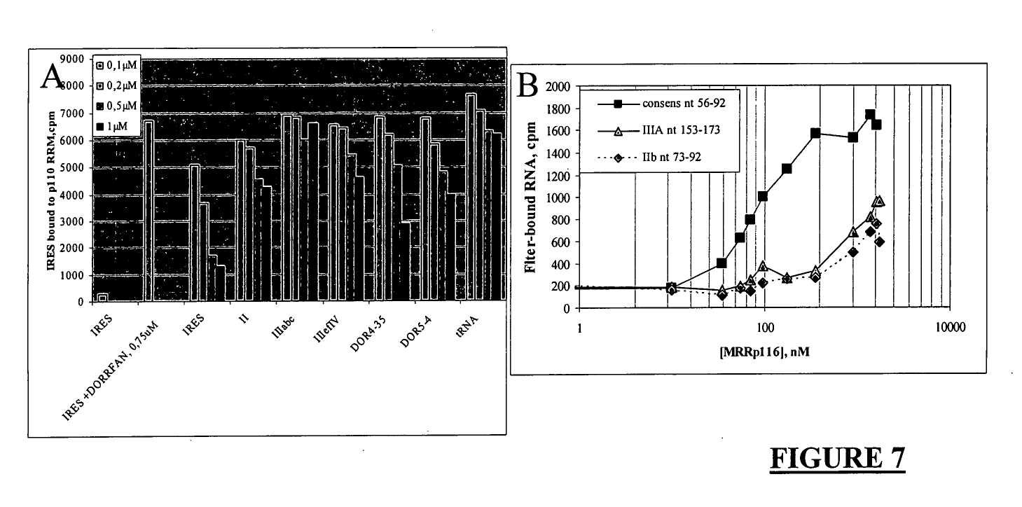

FIG. 7A shows the ability of the consensus sequence (DOR4-35 and DOR5-4) to inhibit the interaction between IRES and RRM of p116, compared with that of the IRES, II, IIabc, IIIefIV and transfer RNA.

FIG. 7 B shows that the consensus sequence (nt 56-92) exhibits an affinity for the RRM-p116 of eIF3 that is greater than that of fragment IIIa (nt 153-173) and than that of the apical portion of fragment II (nt 73-92).

FIG. 8 represents the ability of the aminoglycosides to inhibit the binding of the RRM of eIF3 to the consensus sequence of region II of the IRES of HCV.

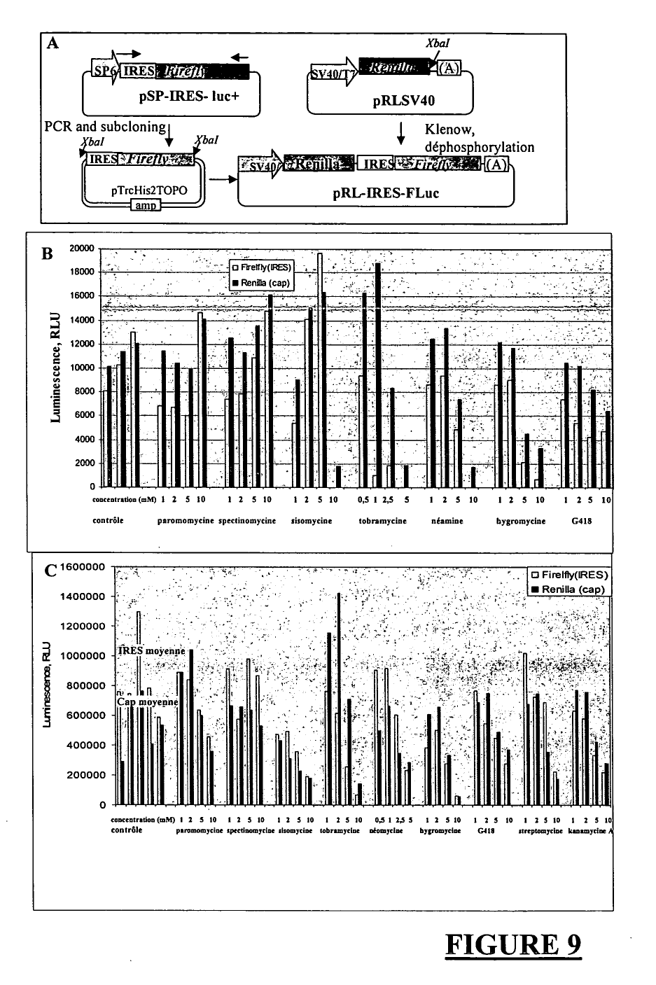

FIG. 9 represents the effect of the aminoglycosides on the cap-dependent and cap-independent translation in cell culture. The bicistronic construct for which the cloning scheme is represented in FIG. 9A is used for the transient transfection of HeLa cells with 1 μg of plasmid DNA (FIG. 9B) and 2.5 μg of pDNA (FIG. 9C).

### EXAMPLE 1

### Demonstration of the Ability of the Recognition Motif (RRM) of p116 to Bind to Region II of the IRES of HCV

1/Cloning and Expression of the Recognition Motif of p116 of eIF3

The amino acid sequence of the recognition motif (RRM) of the p116 protein corresponds to the sequence SEQ ID 5 located between amino acids 175 and 279 of the sequence SEQ ID 4 (corresponding to the sequence of the p116 protein). The cDNA encoding the RRM is amplified by RT-PCR from DNA extracted from HeLa cells, in the presence of the following primers:

| | |
|---|---|
| CATATGGATCGGCCCCAGGAAGCAGATGGAATC: | SEQ ID 6 |
| GTGCTCGAGCCACTCGTCACTGATCGTCATATA: | SEQ ID 7 |

The amplified fragment is cloned into a plasmid pET-30b (Novagen) as a fusion with a C-terminal His₆-Tag between the Nde and Xho sites. The protein is then produced in E. coli (strain BL21lysS) and then purified on Ni2⁺-NTA agarose under native conditions.

2/Synthesis of the Total IRES and its Fragments IIIabc, IIIefIV and Iiab

a/ Principle

Four different nucleotides, respectively:
a nucleotide sequence corresponding to the entire IRES located between nucleotides 40 and 372 of the HCV DNA (b),
a nucleotide sequence corresponding to region IIIabc, located between nucleotides 141 and 252 of the HCV DNA (c),
a nucleotide sequence corresponding to region IIIefIV located between nucleotides 250 and 372 of the HCV DNA (d),
a nucleotide sequence corresponding to region Iiab located between nucleotides 40 and 119 of the HCV DNA (e)
   are synthesized and cloned.

b/ Cloning of the Entire Nucleotide Sequence of the IRES (SEQ ID 1)

The cDNA of the IRES (SEQ ID 1) is amplified by RT-PCR from total RNA isolated from patients who have HCV (genotype 1b), in the presence of the following nucleotide primers:

SEQ ID 8

ACCGCTAGCCTCCCCTGTGAGGAACTACT:

SEQ ID 9

GAAAGCTTTTTTCTTTGAGGTTTAGGATTTGTGCTCATGATGCACG:

The amplified fragment is first cloned into a plasmid pGEM-T and then subsequently into pSP-luc+ (Promega) between NheI and Hind III sites. The plasmid pSP-IRES-luc+ thus obtained contains the IRES of HCV cloned as a fusion with luciferase under the control of the SP6 promoter.

Once sequenced (GenomeExpress, Grenoble), the sequence of the IRES was aligned and compared with the other IRES sequences deposited in the databanks (such as D49374 or AF139594). The identity observed was 96.6%, which corresponds to the average degree of genomic variability of IRESs between various HCV strains.

c/ Synthesis of Region IIIabc

The cDNA of region IIIabc is synthesized in the following way. Two overlapping oligonucleotides, the first of which, SEQ ID 10, consists of the T7 polymerase promoter and of the nucleotide sequence of regions IIIa and IIIb (nt 139-215 of the HCV RNA), and the second of which, SEQ ID 11, consists of the nucleotide sequence of regions IIIb and IIIc (nt 193-252 of the HCV RNA), are hybridized in the presence of a Klenow fragment. The oligonucleotides have the following sequences:

| | |
|---|---|
| TAATACGACTCACTATAGGGTAGTGGTCTGCGGAACCGGTG | SEQ ID 10 |
| AGTACACCGGAATTGCCAGGACGACCGGGTCCTTTCTTGGA | |
| TAAACCCGCTCAA: | |
| TAGCAGTCTCGCGGGGGCACGCCCAAATCTCCAGGCATTGA | SEQ ID 11 |
| GCGGGTTGATCCAAGAAAG.: | |

The double-stranded cDNA fragment obtained is then amplified by PCR in the presence of T7 corresponding to SEQ ID 12: TAATACGACTCACTATAGGG,

and of a flanking oligonucleotide, the sequence of which is as follows:

| | |
|---|---|
| TAGCAGTCTCGCGGGGGCACG.: | SEQ ID 13 |

d/ Synthesis of Region IIIefIV

The cDNA corresponding to region IIIefIV (nt 250-372) was obtained by PCR amplification of the plasmid pSP-AIRES-luc+ using the primers whose nucleotide sequences correspond to those of SP6 (SEQ ID 14: TATTTAGGTGACACTATAGAAT) and SEQ ID 13. The plasmid pSP-ΔIRES-luc+ results from digestion of the plasmid pSP-IRES-luc+ with NheI, the cleavage sites being located between nucleotides 39/40 and 248/249 of the IRES. The SP6→SEQ ID 13 amplification product is then used as a template in the in vitro transcription reaction using SP6 polymerase (SP6 MEGAscript, Ambion).

e/ Synthesis of Region Iiab

The cDNA corresponding to region Iiab was obtained by PCR amplification of the plasmid pSP—IRES-luc+using the primers SP6 (SEQ ID 14) and SEQ ID 15 GTCCTGGTGGCTGCAGGACACTCATAC. The SP6→SEQ ID 15 amplification product is then used as template in the in vitro transcription reaction using SP6 polymerase.

3/Binding of the RRM of p116 to the IRES and its Domains IIIabc, IIIefIV, Iiab

Radiolabeled RNA fragments are obtained by in vitro transcription of the abovementioned templates in the presence of [α-32P]UTP. The RNA fragments are purified in a 6% acrylamide-urea gel and precipitated. The RNA pellets are taken up in 25 mM Tris-HCl, pH 7.4. In order to allow renaturation, the RNA was incubated at 65° C. in the abovementioned buffer for 5-7 min and then slowly cooled to ambient temperature. The renatured RNAs were incubated with increasing concentrations of protein in the same 25 mM Tris-HCl buffer, pH 7.4, at ambient temperature for 5 min.

The mixture of proteins and of RNA is then deposited onto a nitrocellulose membrane washed beforehand with the same buffer. The radioactivity of the filter containing the RNA-protein complexes was measured using a Trilux MicroBeta radioactivity counter (PerkinElmer).

In the event of competitive inhibition, the RRM of p116 was preincubated with nonradiolabeled RNA (concentration: protein 0.7 μM, RNA: 0.1 to 1 μM) for 30 min at ambient temperature, followed by addition of the radiolabeled IRES RNA. The binding of the RNA to the protein was analyzed exactly as above.

4/Results

The affinity of the RNA recognition motifs (RRMs) of the p116 subunit of eIF3 for the whole IRES and its fragments II, IIIabc and IIIefIV was studied by retention on nitrocellulose. As is shown in FIG. 3, the protein binds the IRES with an apparent Kd of 0.8 μM. However, the affinity of RRMp116 for the fragment IIIabc (putative eIF3 binding site) is significantly lower than that for the IRES and comparable to that for IIIefIV used as a negative control. This was expected, all the more so since previously published results assumed that the apical portion of the loop forming region IIIb was the probable eIF3 binding site (Sizova D, 1998; Buratti, 1998; Kieft et al, 2001; F-A-2 815 358). In reality, and as this figure shows, the recognition motif of eIF3 is not found on region IIIabc, but on region II.

### Example 2

### Identification of the Consensus Sequence that Binds to the p116 RRM

1/Production of Random Subfragments of the HCV IRES and Method for Selecting Specific Fragments that Bind to the p116 RRM of eIF3

The method called SERF (Selection of Random Fragments) described by Stelz (2000) is used to synthesize random sequences of the IRES. The principle thereof is represented in FIG. 4.

A/ Production of Subfragments

2 μg of IRES cDNA are digested with 5U of a Dnase I (Rnase-free, Amersham), at ambient temperature for 15 minutes, making it possible to obtain cDNA fragments whose length varies between 30 and 100 nucleotides. Blunt ends are generated at the end of the cDNA fragments obtained, with Taq polymerase at 72° C., for 10 minutes in a PCR buffer based on 1 mmol of dNTP. The Taq polymerase adds additional “dA” residues to the 3′ end of fragments, at the same time (FIG. 4). This makes it possible to increase the ligation efficiency of the fragments obtained, in the vector pGEM-T-Easy (Promega), which in turn has complementary “dTs” at the 5′ end (FIG. 4).

The DNA fragments are then cloned in the presence of T4 DNA ligase (BioLabs), into a vector pGEM-T-Easy (Promega) between the T7 and SP6 promoters. The DNA fragments are then amplified in the presence of the T7 and SP6 oligonucleotides, and the amplification product is then used as a template for transcription with SP6 (MEGAscript, Ambion). The transcript longer than 200 nt corresponding to the transcripts with the insert >60 nt were purified on a 10% acrylamide, 8M urea gel (FIG. 4, M corresponding to “Century markers” RNA markers, Ambion).

B/ Selection of Subfragments

The eIF3 p116 RRM recombinant protein is purified on an Ni-NTA-agarose column under native conditions (FIG. 5). The purified protein is then incubated with the library consisting of the purified RNA fragments obtained above, in a 25 mM Tris-HCl buffer, pH 7.4, for 15 min at ambient temperature. The RNA concentration is, at the start, equal to 0.2 μM and that of the protein is equal to 0.8 μM. The protein/RNA mixture is then deposited onto a nitrocellulose membrane prewashed with the same buffer. The filter containing the RNA-protein complexes is then cut up into pieces and the RNA is extracted with a solution containing 0.1% SDS, 0.3M sodium acetate, pH 5.0, for one hour at ambient temperature. The RNA is then recovered by precipitation from ethanol in the presence of tRNA used to facilitate precipitation. The RNA pellet is then taken up in 10 μl of water and subjected to reverse transcription in the presence of the “Stratascript” reverse transcriptase from the T7 oligonucleotide (Stratagene). The single-stranded DNA fragments are then purified by PCR using the T7 oligonucleotide (SEQ ID 14), the SP6 oligonucleotide (SEQ ID 14) and the sequence SEQ ID 16 corresponding to the linker region adjacent to SP6:
SEQ ID 16: TATTTAGGTGACACTATAGAATACTCAAGCTATGCA TCCAACGCGTTG

A control PCR is performed in parallel, with the SP6 and T7 oligonucleotides, in order to confirm the absence of contaminating template DNA among the selected RNAs. The PCR-amplified fragments are subsequently purified and then used as transcription template in the subsequent cycle. The selection/amplification cycle is repeated 5 times. The RT-PCR products are analyzed on a 2% agarose gel (FIG. 5: Φx are DNA markers (stratagene), 1 and 2 are amplification products obtained with the SP6 or SEQ ID 16 primers. The RNA concentration during the subsequent cycles is equal to 0.058 μM and that of the protein is evenly decreased from a value of 1.2 μM in the second cycle to a value of 0.2 μM in the fifth cycle. The RT-PCR products obtained after the fourth and fifth cycles are cloned into a plasmid pTrcHis2-TOPO (Invitrogen) chosen to facilitate the cloning process in the absence of the T7 promoter. The plasmids were purified and sequenced. The sequences obtained were aligned using the Clustal W DNA program (Thompson, J. D. et al., CLUSTAL W: improving the sensitivity of progressive multiple sequence alignment through sequence weighting, positions-specific gap penalties and weight template choice. (1994) Nucleic Acids Research, 22, 4673-4680), available at the “Pôle Bio-Informatique Lyonnais site.

2/Results

As illustrated in FIG. 6A, among the 16 RNA sequences selected, cloned after 4 and 5 cycles and sequenced using the T7 primer, 11 clones contain the sequence UACUGUCUUCACGCAGAAAGCGUCUAGCAUGGCGUU corresponding to nucleotides 56 to 92 of the sequence SEQ ID 1, 2 clones contain the sequence CGCCTCATGCCTGGAGAT (nt 61-72 of SEQ ID 1) and one clone shows homology with the portion 84-90 of SEQ ID 1. Thus, these results identify the IRES region 56-92 of sequence TACTGTCTTCACGCAGAAAGCGTCTAGCCATGGCGTT (SEQ ID 3) as corresponding to the p116 RRM binding site (FIG. 6B).

The competitive inhibition hypothesis offers an additional means for studying the specificity of the interaction in question. As is indicated in FIG. 7 A, the consensus sequences of the clones 4-35 (DOR 4-35) and 5-4 (DOR 5-4) are the most effective inhibitors (after the IRES itself) of the IRES-p116 RRM interaction. These results confirm that the consensus sequence identified is a determinant in the binding of p116 RRM to the whole IRES.

In addition, the results of the studies of affinity of p116 RRM for the various fragments of the IRES (FIG. 7B) by “filter-binding assay” show that the consensus fragment nt 56-92 is sufficient to allow the binding of p116 RRM. On the other hand, the region 73-92 corresponding to the apical loop of region II (IIb) is not sufficient for the binding of this polypeptide.

### Example 3

### In Vitro Screening Assay

The advantage of the present discovery is that of seeking to inhibit the binding of the RRM of p116 to the consensus sequence SEQ ID 3 of region II of the IRES in order to prevent translation initiation and, consequently, protein synthesis by HCV.

Among the potential molecules, the applicant selected aminoglycosides. Aminoglycosides represent a class of chemical molecules that interact specifically with certain folded RNA molecules, such as 16S ribosomal RNA, ribozymes, and the TAR region of HIV. However, the specificity of these molecules with respect to HCV RNA and also their ability to inhibit the IRES-dependent translation of the HCV IRES has not previously been demonstrated.

The screening assay is carried out as follows. The RRM of p116 and the consensus sequence of region II are incubated in the presence of various aminoglycosides. The RNA mixture is then deposited onto a nitrocellulose membrane under the same conditions as in example 2.

The results are represented in FIG. 8. Among the 15 aminoglycosides tested at 4 different concentrations, the compounds tobramycin and streptomycin inhibit the formation of the RNA-protein complex at all the concentrations tested. Tobramycin inhibits 43% of the p116 RRM/II complex at a concentration of 20 μM, and 54% at 40 μM. Streptamycin, for its part, inhibits 25% of the p116 RRM/II complex at a concentration of 20 μM, and 36% at 40 μM. On the other hand, neomycin and sisomycin only inhibit the complex at concentrations greater than 40 μM.

The aminoglycosides kanamycin A, kanamycin B and tobramycin are molecules that have a very similar structure. However, tobramycin (43% inhibition at 20 μM) is more active than kanamycin B (22% inhibition at 20 μM) which, in turn, is more active than kanamycin A (0% inhibition at 20, 40 and 80 μM). This indicates the presence of an amino group in the R2-position (tobramycin and kanamycin B) promotes dissociation of the complex, whereas the presence of the hydroxyl group in the R1-position is unfavorable thereto (kanamycin A). On the other hand, the amino group in the 6′-position is not directly involved in the interaction and can therefore be used to introduce modifications that make it possible to decrease the active concentrations required.

### Example 4

### Inhibition of the Cap-Independent Translation Ex Vivo

In this example, a correlation is established between the results obtained in the in vitro screening system (example 2) and those of a bicistronic cellular system.

This makes it possible to verify that the inhibition of the formation of the complex of the eIF3 p116 protein/IRES RNA of HCV by a chemical molecule also results in inhibition of the IRES-dependent translation in cells ex vivo. Moreover, this assay makes it possible to detect any possible toxicity of the molecule in question for the cell itself, while at the same time measuring its effect on the cap-dependent translation.

a/ Preparation of Bicistronic Vectors

Bicistronic constructs consisting of a first cistron corresponding to the Renilla luciferase gene, followed by the IRES sequence, followed by a second cistron corresponding to the Firefly luciferase gene (pRluc-IRES-Fluc) are prepared in the following way. A plasmid pRL-SV40 (Promega) is linearized with Xba I and dephosphorylated. In parallel, the IRES is amplified with the Firefly luciferase gene by PCR, in the presence of complementary oligonucleotides containing the Xba I sites. The PCR products are then subcloned in plasmid pTrcHis2-TOPO (Invitrogen) in order to control the digestion. The ligation of the insert containing the IRES with the Firefly luciferase gene and the linearized vector pRL-SV40 is carried out using T4 DNA ligase (Biolabs).

b/ Transfection of HeLa Cells

10⁷ HeLa cells suspended in serum-free DMEM are transfected with 1 to 2.5 μg of plasmid pRluc-IRES-Fluc by electroporation at 0.5 V for 30 milliseconds using a Gene Pulser (BioRad). The cells are then cultured in 24- or 96-well plates in the presence of various aminoglycosides, at concentrations between 2 and 5 mM, for 24-36 h. The Renilla luciferase activity (cap-dependent translation) and that of the Firefly luciferase (cap-independent=viral translation) in the cell lysates is measured and compared by means of the Dual-luciferase assay (Promega) and of a Lumat LB9507 luminometer (Berthold).

c/ Results

The effect of 10 different aminoglycosides on the IRES-dependent translation and on the cap-dependent translation were studied using HeLa cells transfected with the bicistronic construct (FIG. 9A).

According to the results given in FIG. 9B, among the 9 aminoglycosides tested, at a concentration of 1 mM, tobramycin exhibits 90.4% inhibition of the synthesis of the Firefly luciferase controlled by the IRES of the hepatitis C virus, whereas the synthesis of the Renilla luciferase, which is cap-controlled, is not inhibited (168% of the control). A similar effect is observed at a 2 mM concentration of tobramycin (the IRES-luciferase synthesis is 83% inhibited, and that of cap-luciferase is only 27% inhibited).

Hygromycin and G418 inhibit both the cap-dependent and IRES-dependent translation in an IRES-nonspecific manner.

The effect of paramomycin at concentrations of 1, 2 and 5 mM is more pronounced on the IRES-dependent translation (37% inhibited) than on the cap-dependent translation (6.3% inhibited) and is therefore moderately IRES-specific.

When the amount of RNA produced in the cells is increased, using a higher concentration of plasmid DNA (FIG. 9 C), the effect of tobramycin is less pronounced (36.5% inhibition of the IRES-dependent translation at 2 mM, and 69% inhibition at 5 mM), with cap-dependent synthesis not inhibited (268 and 134% of control). Under the same conditions, streptomycin inhibits the translation of the two cistrons in an IRES-nonspecific manner (from a concentration of 5 mM). Thus, some aminoglycosides are capable of inhibiting the IRES-dependent translation in an IRES-specific manner, without inhibiting the host cell's translation. These molecules, which are nontoxic for the cell at the concentrations indicated, can be used to treat hepatitis C. The results obtained with the bicistronic system are coherent with those of the screening assay developed by the applicant (p116 RRM/domain II): the same molecule, tobramycin, was identified as being the most active in the two systems. This shows the relevance of the screening assay claimed, which can be used for identifying novel inhibitors of the binding of eIF3 to the IRES, and of the IRES-dependent translation.

### BIBLIOGRAPHY

1. Choo Q L, Kuo G, Weiner A J, Overby L R, Bradley D W, Houghton M. (1989) Science 244:359-62 “Isolation of a cDNA clone derived from a blood-borne non-A, non-B viral hepatitis genome.”
2. Sizova D V, Kolupaeva V G, Pestova T V, Shatsky I N, Hellen C U (1998) J Virol 72:4775-82, Specific interaction of eukaryotic translation initiation factor 3 with the 5′ nontranslated regions of hepatitis C virus and classical swine fever virus RNAs.
3. Kieft J., Zhou K., Jubin R., Doudna J., RNA (2001), 7:194-206, Mechanism of ribosome recruitment by hepatitis C IRES RNA
4. Buratti E, Tisminetzky S, Zotti M, Baralle F E (1998) Nucleic Acids Res 26:3179-87 Functional analysis of the interaction between HCV 5′UTR and putative subunits of eukaryotic translation initiation factor eIF3.
5. Zhao W D, Wimmer E. (2001) J Virol 75:3719-30 Genetic analysis of a poliovirus/hepatitis C virus chimera: new structure for domain II of the internal ribosomal entry site of hepatitis C virus.
6. Block K L, Vornlocher H P, Hershey J W. Characterization of cDNAs encoding the p44 and p35 subunits of human translation initiation factor eIF3. (1988) J Biol. Chem. 273:31901-8.
7. Asano K, Vornlocher H P, Richter-Cook N J, Merrick W C, Hinnebusch A G, Hershey J W. (1997) Structure of cDNAs encoding human eukaryotic initiation factor 3 subunits. Possible roles in RNA binding and macromolecular assembly. J Biol. Chem. 272:27042-52.
8. Stelz U, Spahn C, Nierhaus K H, (2000) Proc Natl Acad Scie USA 97, 4597-4602 “Selecting rRNA binding sites for the ribosomal proteins L4 and L6 from randomly fragmented rRNA application of method called SERF”

1. A method for screening molecules according to which, in vitro:
a/ the p116 subunit (SEQ ID 4) of the eIF3 protein, the nucleotide sequence of region II (SEQ ID 2) of the HCV IRES or any sequence containing at least 10 successive nucleotides of region II (SEQ ID 2) of the HCV IRES, and the molecule to be tested are incubated together,
b/ the possible formation of p116/IRES region II complexes is then detected, an absence of complex reflecting the inhibitory capacity of the molecule tested, to inhibit the formation of said complexes,
c/ the molecules that inhibit the formation of the complexes are selected.

2. The method as claimed in claim 1, characterized in that only the sequence of the recognition motif of the p116 protein (SEQ ID 5) is incubated.

3. The method as claimed in claim 1, characterized in that only part of region II is incubated and corresponds to the consensus nucleotide sequence SEQ ID 3 or a sequence comprising at least 8 successive nucleotides of the sequence SEQ ID 3.

4. The method as claimed in claim 1, characterized in that the molecule to be tested is incubated at increasing doses.

5. The method as claimed in claim 1, characterized in that the detection is carried out by filtration of the mixture through a nitrocellulose membrane, and then by measurement of the radioactivity attached to the membrane, corresponding to the amount of RNA bound to the membrane.

6. The method as claimed in claim 1, characterized in that the influence of the molecule selected in c) on the cap-independent translation and the cap-dependent translation is then tested, ex vivo, so as to select only the molecules that inhibit the cap-independent translation without influencing the cap-dependent translation.

7. The method as claimed in claim 6, characterized in that bicistronic vectors are constructed, consisting of two luciferases framing the sequence of region II (SEQ ID 2) or any sequence containing at least 10 successive nucleotides of region II (SEQ ID 2), or the consensus sequence (SEQ ID 3) or a sequence comprising at least 8 successive nucleotides of the sequence SEQ ID 3; the first luciferase being translated in a cap-dependent manner and the second in a cap-independent manner, or vice versa.

8-10. (canceled)

11. A pharmaceutical composition comprising an antisense oligonucleotide complementary to the sequence SEQ ID 3 or to any sequence comprising at least 8 successive nucleotides of the sequence SEQ ID 3, with the exception of the oligonucleotides of sequence TAGACGCTTTCTGCGTGAAGACAGTAGT, GAAGACAGTAGTTCCTCACAGGGGA GTG or GCCATGGCTAGACGCTTTCT.

12. (canceled)

13. A method for the treatment of a condition associated with a virus selected from the group consisting of hepatitis C (HVC), classical swine fever (CSFV) or bovine viral diarrhea (BVDV) comprising administering to a mammal in need of such treatment a pharmaceutically effective dose of an aminoglycoside.

14. The method of claim 13, wherein the aminoglycoside is tobramycin.

15. A method for the treatment of hepatitis C (HVC) comprising administering to a mammal in need of such treatment a pharmaceutically effective dose of a pharmaceutical composition comprising an antisense oligonucleotide complementary to SEQ ID NO.: 3 or to any sequence comprising at least 8 successive nucleotides of the sequence SEQ ID NO.: 3, with the exception of oligonucleotides of sequence TAGACGCTTTCTGCGTGAAGACAGTAGT, GAAGACAGTAGTTCCTCACAGGGGA GTG or GCCATGGCTAGACGCTTTCT.
